(19) United States
(12) Patent Application Publication
(10) Pub. No.: US 2011/0046348 A1
(43) Pub. Date: Feb. 24, 2011

(54) METHODS OF PREPARING PEPTIDE DERIVATIVES

(76) Inventor: Marian Kruszynski, King of Prussia, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/988,854

(22) PCT Filed: May 11, 2009
(86) PCT No.: PCT/US09/43423

§ 371 Date: Oct. 21, 2010

Related U.S. Application Data
(60) Provisional application No. 61/053,126, filed on May 14, 2008.

(51) Int. Cl. C07K 2/00

(52) U.S. Cl. 530/332

(57) ABSTRACT

The invention relates to methods of preparing peptide hydrazides useful in as intermediates in preparing derivatized peptides and amenable to conversion to reactive azide comprising species. The invention relates to chemical methods of preparing such species from protected peptide-resins containing the aspartyl or glutamyl residues with orthogonal side-chain carboxylic acid protecting groups ester of Asp and Glu. These esters can be selectively converted to the corresponding side-chain hydrazides useful in various synthetic applications

### PRIOR APPLICATION

This application claims priority to U.S. application Ser. No. 61/053,126, filed May 14, 2008, which is entirely incorporated herein by reference.

### BACKGROUND OF THE INVENTION

1. Field of the Invention

The invention relates to methods of solid-phase and solution phase peptide synthesis for preparing peptide hydrazides useful in as intermediates in preparing derivatized peptides and amenable to conversion to reactive azide comprising species.

2. Description of the Related Art

Protected peptide hydrazides are convenient intermediates because they can be converted easily into the corresponding azides, which can later be used in coupling reactions to prepare large peptides by a convergent approach (Lloyd-Williams et al. Tetrahedron 49: 11065, 1993). Ligation by hydrazone formation is a commonly used method in protein and carbohydrate chemistry (Bergbreiter & Momongan 1991, In Comprehensive organic synthesis and efficiency in modern organic chemistry (ed. B. M. Trost and J. Flemming), Vol. 2, p. 503, Pergamon, New York) and acylhydrazine-aldehyde chemistry has been applied successfully for site-specific conjugation to proteins, protein semi-synthesis, and backbone engineering (Gaertner, et al. Bioconjugate Chem. 3: 262, 1992)(Gaertner et al. J. Biol. Chem. 269: 7224, 1994) (Fisch et al. Bioconjugate Chem. 3: 147, 1992).

Azide coupling is another method used in peptide synthesis that has the advantage of producing minimal concomitant racemization. Peptide azides are usually generated from the corresponding peptide hydrazides and coupled at low temperatures.

Tert-Butyl ester (Roeske Chem. Ind. (London), (1059), 1121) (Anderson & Callahan, J. Am. Chem. Soc. 82: 3359, 1960) protecting groups are among the most useful in peptide synthesis, since the treatment of protected peptides with mild acids generally causes fewer side reactions. Compared to primary alkyl esters, tert-butyl esters offer a degree of steric shielding which make them resistant to attack by a wide range of nucleophiles. The α-tert-butyl esters group has been used because of its resistance to alkaline hydrolysis, hydrazinolysis, aminolysis, and hydrogenolysis (In Methods of Organic Chemistry (Houben-Weyl) Synthesis of Peptides and Peptidomimetics, Georg Thieme Verlag Stuttgart—New York 2004, Volume E 22, p. 209. Since the inception of 9-fluorenylmethoxycarbony (Fmoc) solid phase peptide synthesis (SPPS), Asp and Glu carboxylic acid side chains have been successfully protected by the tert-butyl (tBu) group (Chang et al. Int. J. Peptide Protein Res. 15: 59-66, 1980) and readily deprotected using strong acied (TFA) (Fields & Noble, Int. J. Peptide Protein Res. 35: 161-214, 1990). The mechanism of deprotection of functional groups by acidolysis was described in details (In Chemistry of Peptide Synthesis. N. Leo Benoiton, Taylor & Frabcis Group, CRC Press 2006, p. 71).

Hydrazinolysis is not compatible with Boc/Bzl chemistry, as benzyl esters react readily with hydrazine. However, some papers reported the hydrazine cleavage of the blocked peptides from the Merrifield resin (Merrifield, J. Am. Chem. Soc. 85: 2149, 1963; Merrifield, Biochemistry 3: 1385, 1964). Kessler and Iselin have shown that side reactions causing low yields accompanies hydrazine cleavage of a peptide from the Merrifield polymer (Kessler & Iselin, Helv. Chim. Acta 49: 1330, 1966). This method was used for the preparation of water-insoluble, blocked peptide hydrazides which do not contain side-protected aspartic or glutamic acid residues or other groups used in Boc chemistry that are labile to hydrazine (Ohno at al. J. Am. Chem. Soc. 89: 5994-5995, 1967). The urethane-based protecting groups amino functions: Z, Boc and other related tert-alkyl urethane derivatives; and Trt and Tos derivatives are stable to hydrazine (Lloyd-Williams et al. Tetrahedron 49: 11065, 1993). The ether-type protecting groups of hydroxyl functions, e.g. tert-butyl, benzyl, halobenzyl ethers are stable, whereas the S-tert-butyl, S-acetamidomethyl and S-benzyl cysteine derivatives are partially sensitive to hydrazine treatment (Lloyd-Williams, 1993).

Peptide hydrazides can be obtained by hydrazinolysis of peptide benzyl esters attached to resins, as well as to 2-methoxy-4-alkoxybenzyl alcohol resin (SASRIN™)-supported peptides. Protected peptide hydrazides can be synthesized with 4-alkoxybenzyloxycarbonyl-hydrazide resin (Wang, J. Am. Chem. Soc. 95: 1328, 1973), Trt(2—Cl)-hydrazine resin (Vliet et al. In Peptides 1992, Schneider, C. H.; Eberle A. N., Eds.; ESCOM: Leiden, pp 279-280, 1993) (Stavropoulos et al. Lett. Pept. Sci., 2: 315 (1995) or Ddz-hydrazine BAL resin (Royo et al. React. Funct. Polym., 41: 103 (1994). The last system also allows the preparation of bis-peptide hydrazides. Peptide intermediates containing Glu(OtBu) residues are readily converted into the corresponding C-terminal hydrazides (Kappeler & Schwyzer, Helv. Chim. Acta 44: 1136, 1961; Schwyzer, Helv. Chim. Acta 44: 1991, 1961). In the case of Asp(OtBu) residues results were reported depending upon the reaction conditions and peptide sequence (Scoffone & Marchiori, Gazz. Chim. Ital. 94: 695, 1964; Schroder, Justus Liebigs Ann. Chem. 681: 231, 1965; Ondetti et al. Biochemistry 7: 4069, 1968; Schwyzer et al. Helv. Chim. Acta, 46: 1975, 1963).

In the case of the base-sensitive Asp-Gly and Asp-Ser sequences, the tert-butyl ester protection does not prevent aspartimide formation (Bernhard et al. J. Am. Chem. Soc. 84: 2421, 1962). In Fmoc/tBu based SPPS, the repetitive piperidine treatment needed for Fmoc removal lead to aspartimide formation. This side-reaction involves attack of the nitrogen attached to the α-carboxy group of aspartic acid or asparagines on the side-chain ester or amide group respectively, resulting in formation of a five-member imide ([mass: M−18 Da]+). This intermediate can suffer a number of fates: it can undergo ring opening with piperidine during Fmoc-removal, leading to formation of the corresponding α- and β-piperidides ([mass: M+67 Da]+), or it can survive cleavage from the resin, to later hydrolyse in solution, giving the corresponding α- and β-aspartyl peptides. The reaction is highly sequence dependent, but occurs most frequently with peptides containing the Asp(OtBu)-X motif, where X=Asn(Trt), Gly, Ser, Thr (Lauer et al. Lett. Pept. Sci., 1: 197, 1994; Dolling et al. J. Chem. Soc., Chem. Commun., 1989: 853). Similarly, in cases of Asn-Gly sequences α→β transpeptidation on treatment with hydrazine was found to occur (Roeske Chem. Ind. (London), (1059), 1121; Jenkins et al. J. Am. Chem. Soc. 91: 505, 1969).

Previously, it was shown that in solution, that peptide intermediates containing Glu(OtBu) residues were converted into the corresponding hydrazides (Kappeler & Schwyzer, Helv. Chim. Acta 44: 1136, 1961; Schwyzer & Kappeler, Helv. Chim. Acta 44: 1991, 1961). In particular, the reaction of Z-Glu(OtBu)-His-OCH₃ with hydrazine hydrate to give Z- Glu(OtBu)-His-NH—NH₂was described.

In another example, Asp(OtBu) residues were reported to undergo hydrazinolysis depending upon the reaction conditions and peptide sequence. N-Benzyloxycarbonyl-L-aspartyl dihydrazide was formed from the Boc-amine protected monomer or from a Boc-Met-Asp(OtBu)-OCH3 (Scoffone & Marchiori, Gazz. Chim. Ital. 94: 695, 1964). Thus, it has not been established under what conditions or in what environments the tert-butoxylated carboxyl side chain will undergo hydrazinolysis.

It would be of tremendous benefit in the practice of peptide synthesis to be able to control the formation of side chain hydrazide.

### SUMMARY OF THE INVENTION

The invention relates chemical methods useful in protected-peptide synthesis to convert the orthogonal side-chain carboxylic acid protecting groups ester of Asp and Glu to beta- and gamma-hydrazides, respectively. The invention relates to the identification of conditions for selective formation of an internal beta- and gamma-hydrazides in a protected peptide wherein the protecting group of one or more side chains of Asp and Glu are selected from the group consisting of tert-butyl (tBu), 3-methyl-pent-3-yl (Mpe), and 2-phenyl-isopropyl (PhiPr) and the protected peptide is contacted with hydrazine. The method of the invention comprises the step of subjecting a protected-peptide comprising at least one esterified residue containing a side-chain carboxylic acid selected from the group consisting of Asp(OtBu), Glu(OtBu), Asp(OMpe) or Glu(OMpe); or Asp(OPhiPr) or Glu(OPhiPr) to hydrazinolysis and obtaining a protected peptide-Asp(NH—NH2) or -Glu(NH—NH2) which is not at the C- or N-terminal residue. The method of the invention comprises the step of subjecting a protected-peptide, where the protecting group may be Boc or other group stable to hydrazinolysis, to hydrazinolysis to yield a Boc-protected peptide having at least one side chain carboxy-hydrazide and an N-terminal carboxy-hydrazide. In one aspect of the method of the invention the protected peptide is a Boc-protected peptide-Asp/Glu(OtBu))-peptide′ on a SASRIN resin subjected to hydrazinolysis to form Boc-protected peptide-Asp/Glu (NH—NH₂)-protected peptide′—CO—NH—NH₂ followed by acidolytic cleavage to yield the peptide-Asp(NH—NH₂)-Glu(NH—NH₂)-Peptide′—CO—NH—NH₂.

In another embodiment of the invention, a protected peptide is synthesized having a site specific -Asp(NH—NH₂) or -Glu(NH—NH₂) by directing hydrazide formation at residues having orthogonal side-chain carboxylic acid protecting groups ester of Asp and Glu which are less stable to nucleophilic attack by hydrazine than the orthogonal side-chain carboxylic acid protecting groups ester of other Asp and Glu residues where OPhiPr is less stable than OtBu and OtBu is less stable than OMpe. The invention further relates to conditions and protecting groups useful for the solid phase hydrazinolysis of protected peptides attached to benzylhydrylamine-based (e.g. RINK) or benzyloxycarbonyl-based (e.g. WANG) resins which upon acidolytic cleavage, produce multiple Asp- or Glu-containing peptides with a single, side-specific hydrazide modification and having a C-terminal amide or free C-carboxyl group. The method of the invention encompasses subjecting a Boc-protected peptide-Asp(OtBu)-Asp(OMpe)-protected peptide′-Rink (Wang)-resin to hydrazinolysis to form a Boc-protected peptide-Asp(NH—NH₂)-Asp(OMpe)-protected peptide′-Rink (Wang)-resin which may be converted to a Peptide-Asp(NH—NH₂)-Asp-Peptide′—CO— NH₂ (or COOH; acidolytic cleavage). In one embodiment the protected peptide is of the formula: peptide-Asp/Glu(OtBu)—Xₙ-Asp/Glu(OMpe)-protected peptide′-SASRIN resin where X is any amino acid residue and n can be 0 or any length desired, and the protected peptide is subjected to hydrazinolysis to provide a Boc-protected peptide-Asp/Glu(NH—NH₂)—Xn-Asp/Glu(OMpe)-protected peptide′—CO—NH—NH₂ which may be further converted to Peptide-Asp(NH—NH₂)-Asp-Peptide′—CO—NH—NH₂ by acidolytic deprotection.

In another aspect of the invention, the peptide-hydrazide synthesized using the process of the invention where the hydrazide is not at the alpha carboxyl, is used to ligate the peptide by hydrazone formation. The peptide-hydrazide may be ligated to any desired chemically synthesized structure or group having a suitable reactive radical such as an aldehyde or ketone including but not limited to proteins and peptides, chromophores or flourophores, chelating groups, or itself In a particular embodiment of the use of the peptide-hydrazide produced by the process of the invention, the hydrazino-group is converted to an azide for reaction with amino or amine, aldehyde, or ketone groups for, e.g. multimerization or cyclization.

### BRIEF DESCRIPTION OF THE SEVERAL VIEWS OF THE DRAWING

FIG. 1. An analytical, RP-HPLC (C18) tracing of the crude products of hydrazinolysis of a protected GLP-1 analog peptide-SASRIN (I) and acidolytic deprotection: IIA (2 hours reaction) and IIB (24 hours reaction).

FIG. 2: ¹H-NMR of the GLP-1[D-Ala², Gly³¹-PEG₁₂-Gly³²]-NH—NH₂ (B) in d₆-DMSO showing a lack of -OMe signals at 3.3-3.5 ppm for the potential modification expected to give Asp(OMe)⁹, Glu(OMe)^{3,15}.

FIG. 3. LC-MS analysis of the crude product isolated after 2 hour hydrazinolysis of Boc-Phe-Asp(OtBu)-Lys(Boc)-Asp(OMpe)-Phe-Ala-Phe-Gly-SASRIN-Resin (V).

FIG. 4: LC-MS analysis of the crude product after 24 hour hydrazinolysis of Boc-Phe-Asp(OtBu)-Lys(Boc)-Asp(OMpe)-Phe-Ala-Phe-Gly-SASRIN-Resin (V) showing the two products were identified (LC MS) and quantified (HPLC): VIB (HPLC: 16%, (1259.5 Da) Boc-Phe-Asp(NH—NH2)-Lys(Boc)-Asp(OMpe)-Phe-Ala-Phe-Gly-NH—NH₂ and VID (HPLC: 84%, 1188.3 Da) Boc-Phe-Asp(NH—NH₂)-Lys(Boc)-Asp(NH—NH₂)-Phe-Ala-Phe-Gly-NH—NH₂.

FIG. 5. The LC-MS of the crude VIII resulted from acidolytic treatment of protected peptide mixture VIIA.

FIG. 6. HPLC purified VIIB (Phe-Asp(NH—NH₂)-Lys-Asp(COOH)-Phe-Ala-Phe-Gly-NH—NH₂; calculated MW: 974 Da): A) Analytical RP HPLC (C18) (214 nm); B & C) LC-MS; D) Capillary Electrophoresis (CE) shows a baseline separation of two diastereoisomers LDLLLLL and LLLLLLL and indicate a significant epimerization of the Asp(hydrazide) asymmetric carbon atom.

FIG. 7. HPLC purified VIID, Phe-Asp(NH—NH₂)-Lys-Asp(NH—NH₂)-Phe-Ala-Phe-Gly-NH—NH₂; calculated MW: 988 Da: A) Analytical RP HPLC (C18) (214 nm); B & C) LC-MS; D) Capillary Electrophoresis (CE) shows a separation of four diastereoisomers LDLLLLL, LDLDLLL, LLLDLLL and LLLLLLL and indicate a significant epimerization of the both Asp(hydrazide)s asymmetric carbon atoms.

### DETAILED DESCRIPTION OF THE INVENTION

### Abbreviations

AOC amyloxycarbonyl

BAL backbone amide linker

Boc tert-butyloxycarbonyl

tBu tert-butyl

Bzl benzyl

CE capillary electrophoresis

Da Dalton

Ddz 2-(3,5-dimethoxyphenyl)prop-2-yloxycarbonyl

DIPEA diisopropylethylamine

DMF dimethylformamide

DMSO dimethyl sulfoxide

DPPA diphenyl phosphorazidate

DTT dithiothreitol

Fmoc 9-fluorenylmethoxycarbonyl

Fmoc-Phe-Ser(Ψ^{Me-Me}pro)—OH pseudoproline dipeptide

N³-[Fmoc-Phe]-Oxa(2,2-Me₂) N³-[N-α-Fmoc-Phe-]-2,2-dimethyl-oxazolidine-carboxylic acid

Fmoc-Phe-Thr(Ψ^{Me-Me}pro)—OH pseudoproline dipeptide;

N³-[Fmoc-Phe]-Oxa(2,2,5-Me₃) N³-[N-α-Fmoc-Phe]-2,2,5-trimethyl-oxazolidine-4-carboxylic acid

HCl hydrochloric acid

LC MS liquid chromatography—mass spectrometry

MALDI matrix assisted laser desorption ionization

MAP multiple antigenic peptides

MPTA dimethylphosphorothioyl azide

MS mass spectrometry

MS/MS tandem mass spectrometry

OMe methoxy

OMpe O-(3-methyl)-pent-3-yl

O—2-PhiPr O-(2-phenylisopropyl

Ser(Ψ^{Me-Me}pro) serine-derived oxazolidine-4carboxylix acid

Oxa(2,2-Me₂) 2,2,-dimethyl-oxazolidine-4-carboxylic acid

Thr(Ψ^{Me-Me}pro) threonine-derived oxazolidine-4-carboxylic acid

Oxa(2,2,5-Me₃) 2,2,5 -trimethyl-oxazolidine-4-carboxylic acid

OtBu tert-butoxy

PEG polyethylene glycol

PVDF poly(vinylidene fluoride)

Rink resin trialkoxybenzhydrylamine resin

RP-HPLC reversed-phase high-performance liquid chromatography

SASRIN™ 2-methoxy-4-alkoxybenzyl alcohol resin

SPPS solid phase peptide synthesis

TASP template-assembled synthetic proteins

TBA+NO₂⁻tetrabutylammonium nitrite

TEA triethylamine

TFA trifluoroacetic acid

TIPS triisopropylsilyl

TIS triisopropylsilsne

Tos tosyl

Trt trityl

Wang resin 4-alkoxybenzyloxycarbonyl-hydrazide resin

Z benzyloxycarbonyl

### Definitions

For convenience in describing this invention, the conventional abbreviations for the various amino acids are used. They are familiar to those skilled in the art. All chiral amino acid residues referred to herein are of the natural or L-configuration unless otherwise specified. All peptide sequences mentioned herein are written according to the usual convention whereby the N-terminal amino acid is on the left and the C-terminal amino acid is on the right. Where either of the naturally occurring acidic alpha-amino acids (Asp or Glu) are meant, the term Asp/Glu will be used.

As used herein, the term “esters” refers to esters of a carboxyl group of the polypeptide formed with straight or branched chain saturated alkyl or aryl alcohols.

As used herein the term “amides” refers to amides of a carboxy group of the polypeptide formed with ammonia, or with primary or secondary amines having up to 12 carbon atoms such as for example dimethylamine, diethylamine, di(n-butyl)-amine, n-hexylamine, piperidine, pyrrolidine, morpholine, di(n-hexyl)amine, N-methylpiperazine and the like. Included amides are the naturally occurring amino acid amide of Asp and Glu, Asn and Gln, respectively.

“N-acyl derivatives” refer to those derivatives of an amino group of the polypeptide formed with acyl moieties (e.g. alkanoyl or carbocyclic aroyl groups), such as formamides, acetamides, benzamides, and the like.

“O-acyl derivatives” refer to those derivatives of a hydroxyl group of the polypeptide chain formed with acyl moieties (e.g. alkanoyl or carbocyclic aroyl groups), such as formates, acetates, propionates, benzoates, and the like.

The term “orthogonal” or “orthogonality” when used in reference to side chain protecting groups refers to a situation as described herein in which there are two or more classes of protecting groups on a molecule, each class most optimally removed under specific conditions, while remaining stable to conditions used to remove protecting groups in other classes. Thus, one can remove all protecting groups of one class, while leaving all others intact.

By “protected-peptide” or “peptide” is meant a polyamino acid structure linked by amide linkages wherein reactive side chain residues are reversibly modified in a manner that allows the restoration of the “original” or “natural” structure and reactivity of the side chain.

### Overview

The most common procedure for preparation of protected peptide hydrazides is hydrazinolysis of the corresponding methyl, ethyl or benzyl esters with hydrazine hydrate. Alternatively, hydrazides are obtained by hydrazinolysis of C-terminally activated amino acids and peptides. Hydrazinolysis occurs with similar efficiency when peptides are bound to resin via suitable linkers. A synthetic strategy was developed for solution chemistry that is based on the use of N′-protected amino acid hydrazides (Hofmann et al. (1950) J. Am. Chem. Soc. 72, 2814; Hofmann et al. (1952) J. Am. Chem. Soc. 74, 470). Different hydrazide linkers for polystyrene resins were developed by Wang and Merrifield (Wang, S. S., Merrifield, R. B. (1969) J. Am. Chem. Soc. 91, 6488; Wang, S. S. (1973) J. Am. Chem. Soc. 95, 1328; Wang, S. S. (1975) 40, 1235). After completion of the peptide synthesis on the solid support, the hydrazides can be cleaved from the resin with 50% TFA in 30 minutes.

It was heretofore generally believed that Fmoc/t-butyl protection-based solid phase peptide synthesis on 2-methoxy-4-alkoxybenzyl alcohol resin (SASRIN™) can produce readily fully protected peptide fragments from which peptide hydrazides can be obtained in good yield and purity via cleavage with hydrazine hydrate or hydrazine. The dry peptide resin is merely suspended in DMD or DMA (18 mL/g resin) and left to swell. Then hydrazine hydrate or hydrazine is added (2 mL/g resin) to obtain a 10% solution. After 2-24 hours reaction the resin is filtered off and rinsed with DMF, then product is precipitated by addition of water. The cleavage time has to be optimized individually. In general the tent-butyl esters, as used in the Z/tBu strategy in solution, are stable towards hydrazinolysis (Mergler & Nyfeler, 1991).

The present invention is based on applicants observation and investigation of the additional species recovered with the C-terminal peptide-hydrazide after hydrazinolysis of peptide′ containing tert-butoxycarbonylated acidic residues (Asp(OtBu) and Glu(OtBu)). Side chain beta- and gamma-hydrazides were formed at the positions of the tert-butoxycarbonylated Asp or Glu. Further investigation by applicants confirmed the relative reactivity of various protecting group esters to hydrazinolysis, making the strategic synthesis of side-specific peptide hydrazides in multiple Asp or Glu containing peptide products, possible. For example, the -Asp(OMpe)- residue has higher stability than the corresponding -Asp(OtBu)- ester in reaction with 10-20% hydrazine/DMF, and the -Asp(OtBu)- ester has higher stability than the corresponding -Asp(OPhiPr)- ester.

### General Methods of Peptide Synthesis

In general, these methods comprise the sequential addition to a growing chain of one or more amino acids or suitably protected amino acids. Normally, either the amino or carboxyl group of the first amino acid is protected, by a suitable protecting group. The protected or derivatized amino acid can then be either attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide.

A method for synthesizing the peptides of the present invention is the so-called “Merrifield” solid phase synthesis technique which is well known to those skilled in the art and is set forth in detail in the article entitled “Synthesis of a Tetrapeptide” by R. B. Merrifield, Journal of the American Chemical Society, Vol. 85, pp. 2149-2154 (1963). In this method, a peptide of desired length and sequence is produced through the stepwise addition of amino acids to a growing peptide chain which is bound by a covalent bond to a solid resin particle.

For the correct assembly of peptide sequence, the N^{α}-amino protecting group should be specifically cleavable while leaving the side-chain protecting groups intact (“orthogonal” protection). Other reactive functional groups that require mandatory protection are side-chain amine (Lys), carboxylic acid (Asp, Glu), and the thiol (Cys) groups; protection of hydroxyl (Ser, Thr, Tyr), guanidine (Arg), imidazole (His), and the indole (Trp) groups while optional, is often preferred for minimizing the formation of side products. The overall selection of protecting groups is dictated by the synthetic strategy.

Among the classes of amino protecting groups useful for stepwise synthesis of polypeptides are: (1) acyl type protecting groups illustrated by the following: formyl, trifluoroacetyl, phthalyl, toluenesulfonyl (tosyl), benzensulfonyl, o-nitrophenylsulfenyl, tritylsulfenyl, o-nitrophenoxyacetyl, chloroacetyl, acetyl, γ-chlorobutyryl, etc.; (2) aromatic urethan type protecting groups illustrated by benzyloxycarbonyl and substituted benzyloxycarbonyl such as p-chlorobenzyloxycarbonyl, p-nitrobenzyloxycarbonyl, p-bromobenzyloxycarbonyl, p-methoxybenzyloxycarbonyl, 2-(p-biphenylyl)isopropyloxycarbonyl, 2-benzoyl-1-methylvinyl; (3) aliphatic urethan protecting groups illustrated by tert-butyloxycarbonyl, tert-amyloxycarbonyl diisopropylmethoxy-carbonyl, isopropyloxycarbonyl, ethoxycarbonyl, allyloxy-carbonyl; (4) cycloalkyl urethan type protecting groups illustrated by cyclopentyloxycarbonyl, adamantyloxycarbonyl, cyclohexyloxycarbonyl; (5) thio urethan type protecting groups such as phenylthiocarbonyl; (6) alkyl type protecting groups as illustrated by triphenylmethyl (trityl) and benzyl; and (7) trialkylsilyl groups such as trimethylsilyl.

Preferred protecting groups are tert-butyloxycarbonyl (t-BOC), and tert-amyloxycarbonyl (AOC).

Among the classes of carboxyl protecting groups useful for stepwise synthesis of polypeptides are: (1) substituted or unsubstituted aliphatic ester protecting groups such as methyl, ethyl, t-butyl, 2,2,2-trichlorethyl and t-butyl esters; (2) aralkyl ester protecting groups such as benzyl, p-nitrobenzyl, p-methoxybenzyl, diphenylmethyl or triphenylmethyl (trityl) esters; (3) N-substituted hydrazides such as t-butyloxycarbonylhydrazides and carbobenzyloxycarbonylhydrazides; (4) amide protecting groups formed by condensation of a carboxyl moiety with e.g. ammonia, methylamine, ethylamine, diphenylmethylamine; and the like.

Hydroxyl groups of amino acids such as serine, threonine and hydroxyproline may be protected as aralkyl ethers such as benzyl or tBu ethers.

Suitable solid supports useful for the above synthesis are those materials which are inert to the reagents and reaction conditions of the stepwise condensation-deprotection reactions, as well as being insoluble in the media used. Such resins are known to those skilled in the art. Materials that may be used include, for example, crosslinked polystyrene divinylbenzene resins, crosslinked polyamide resins, polyethyleneglycol resins, appropriately functionalized glass beads, and the like. Resins suitable for solid phase peptide synthesis useful in the method of the invention include SASRIN, RINK, and WANG resins.

### Hydrazinolysis of Resin-Bound Peptides

Solid-phase synthesis of protected peptide segments and the detachment of these from the resin in such a way that the N^{α}-protecting group, in addition to those of the side-chain functional groups, are retained and the C-terminal of the segment is either the free carboxylic acid or a derivative suitable for coupling, are the basic stages of convergent solid-phase synthesis (CSPPS) (Lloyd-Williams et al., 1993). Protected peptide segments are usually chosen so that either Gly or Pro is at the C-terminus in order to avoid epimerization at the C-terminal amino acid. For the synthesis of protected peptide segments, the peptide must be detached from the resin under conditions, which do not provoke the premature deprotection of any of the protecting groups. If the Fmoc/tBu strategy is used then cleavage of the peptide from the resin usually cannot be performed under basic conditions and mild acidolysis is the method of choice. In linear SPPS using Merrifield-type resins, the protected peptide-segments may be detached from such resins by hydrazinolysis of the C-terminal-peptide benzyl ester linkage (Keiser, W., Iselin, B. Helv. Cim. Acta 1966, 49, 1330-1344; Ohno, M., Anfinsen, C. B. J. Am. Chem. Soc. 1967, 89, 5994-5995; Visser, S. et al. Recl. Trav. Chim. Pays-Bas, 1968, 87, 559-571; Murakami, Y. at al. Bull. Chem. Soc. Japan 1978, 51, 2690-2697; Kaufmann, K.-D., Bauschke, S. Z. Chem. 1980, 29, 145-146). The SPPS on 2-methoxy-4-alkoxybenzyl alcohol resin (SASRIN™) can produce readily fully protected peptide fragments from which peptide hydrazides can be obtained in good yield and purity via cleavage with hydrazine hydrate or hydrazine. Protected peptide C-terminal hydrazides so generated may be converted into the azides for subsequent coupling reactions. Wang (Wang, S. S., J. Org. Chem. 1975, 40, 1235-1239) prepared several protected peptide C-terminal hydrazides by subjecting the peptide-resin to hydrazinolysis.

### Stability and Selectivity of Protecting Groups Towards Hydrazinolysis

Commercially available N-α-Fmoc-(O—3-methyl-pent-3-yl)aspartic acid (Fmoc-Asp(OMpe)—OH) has been reported to be more stable to TFA than Fmoc-Asp(OtBu)—OH.

Compared to -tBu ester, the -Mpe ester offers a degree of steric shielding capable of providing more resistance to attack by a wide range of nucleophiles, including hydrazine. The stability of a second commercially available derivative, N-α-Fmoc-(β-2-phenylisopropyl ester) aspartic acid (Fmoc-Asp(O—2-Ph-iPr)—OH (Novabiochem) to reaction with hydrazine was additionally tested.

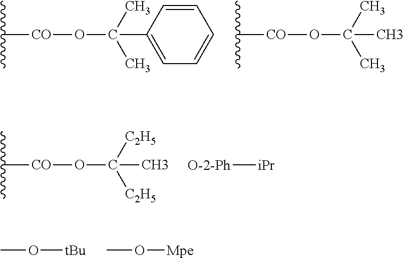

The three protecting groups; O—2-Ph-iPr , —O-tBu, and —O-Mpe were compared for lability under hydrazinolysis using an amphipathic peptide Phe-Asp-Lys-Asp-Phe-Ala-Phe-Gly (IV). After two hours in the presence of hydrated hydrazine, the difference in stability between the esters was clearly demonstrated by examining the relative abundance of the possible di- or tri-hydrazide species (one or two side chain positions in addition to the C-terminal hydrazide.

The carboxyl protecting groups —O-Ph-iPr and —O-tBu were compared experimentally for relative stability under the same reaction conditions using a protected model peptide-resin synthesized with both protecting groups via Fmoc chemistry and subjected to hydrazinolysis. The results described herein, show that the -Asp(OtBu)- ester is more stable in reaction with hydrazine than -Asp(Oph-iPr)-.

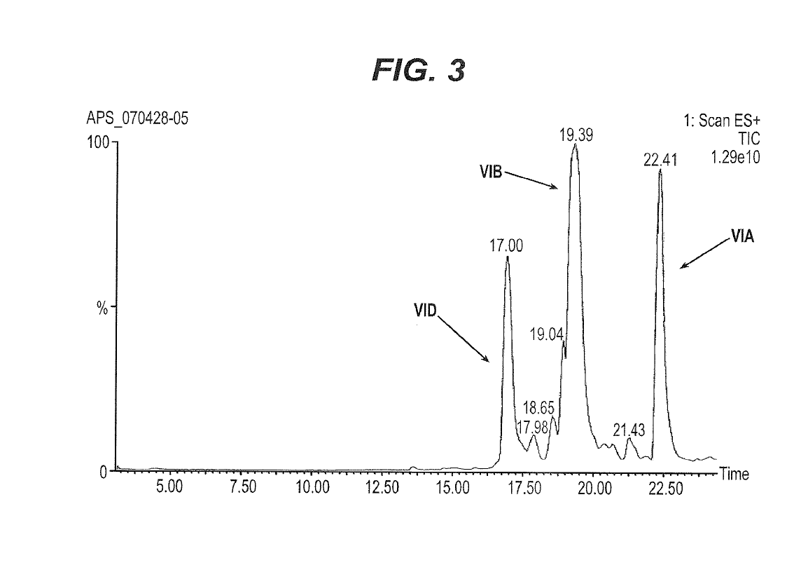

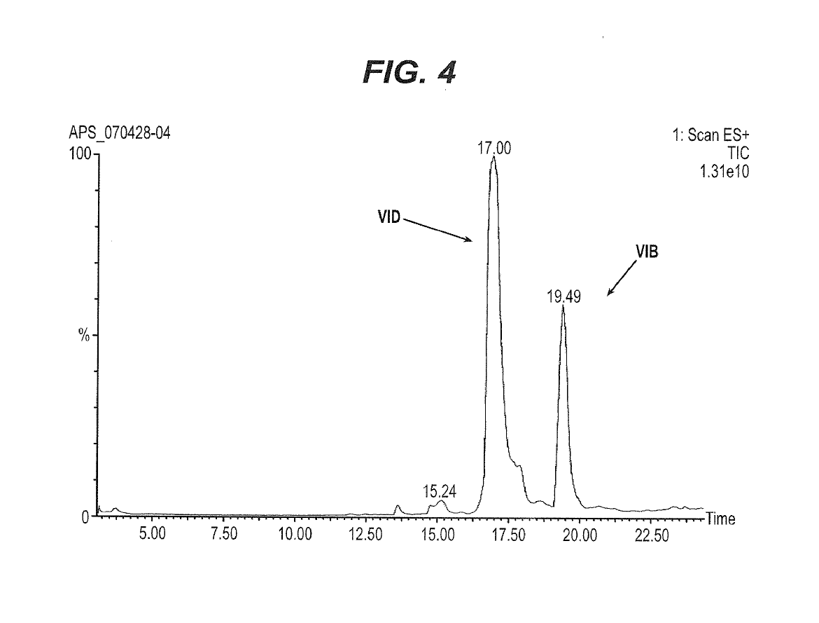

The protection by -Asp(OMpe)- against reaction with hydrazine was observed even after 24 hours reaction (approx. 16% was still remaining) Presented LC-MS data in FIG. 3 and FIG. 4 confirmed the higher selectivity of OMpe ester over OtBu ester protecting group in reaction with hydrazine. The -Asp(OMpe)- residue was more stable than corresponding -Asp(OtBu)- ester in reaction with 20% hydrazine/DMF.

In summary, the applicants have shown that the -Asp(OMpe)- residue is more stable than corresponding -Asp(OtBu)- ester in reaction with 20% hydrazine/DMF and the -Asp(Oph-iPr)- is less so. Therefore, in terms of relative stability to hydrazinolsysis:

OPh-iPr<OtBu<OMpe

These findings enable the design of protected-peptides for the synthesis and isolation of side-specific peptide hydrazides in multiple Asp or Glu containing products, possible.

### Utility

The synthesis of peptides or peptide derivatives having the capacity to participate in additional side-chain linking reactions has many potential applications including but not limited to the formation of multimerized constructs or cyclization of the peptide, derivatization or conjugation to reporter moieties such as biotin, a chromophore or a fluorophor, or to a chelating group, or bioactive such as a toxin or therapeutic agent. While some derivatives can be added during peptide synthesis, some with labile structures may be advantageously attached post-synthesis. For example, functionalities comprising carboxylate groups, such as metal chelating groups with multiple carboxylate moieties, could not be effectively incorporated during the synthetic process.

### 1. Chemoselective Ligation

The principle of chemoselective ligation is based on complementary reactivity in the fragments to be joined, e.g., with aldehyde, R—CHO and hydrazide NH2NH—R′ for selective formation of hydrazone bond, R—C═N—NH—R′. Chemoselective ligation methods permit the condensation of completely unprotected, multifunctional peptide fragments in aqueous media:

Peptide-CHO+H₂NNH-peptide′→Peptide-C═N—NH-peptide′

In one particularly use of the methods described herein, a peptide or peptide′ is synthesized with selectively located side-carboxylic chains converted to hydrazides is used in TASP (template-assembled synthetic proteins) design where it is desirable to have multiple reactive groups (Tuchscherer and Mutter, IN: Peptides and Peptidometics. Ch. 13. Volume E22d, M. Goodman and Felix, A. Eds. Thieme Publishers, New York, 2004).

The present invention therefore provides a means for stable cross-linking of peptides, chemical ligation, internal cyclization, or for building of complex structures. Thus, either a naturally occurring Glu/Asp can be selectively converted to a hydrazine or, subsequently, an azide for reaction with an aldehyde or ketone positioned on a side-group within the chain or at a terminal residue. Methods of forming aldehydes include the use of chemical or enzymatic reactions, e.g. lysyl oxidase will convert the episilon amino group of lysine to an aldehyde.

Multimerization of peptides has long been recognized as a valuable approach to amplify peptide immunogens. Peptides are presented as a larger construct in the form of a multiple antigen peptide (MAP). In this type of construct various copies of the peptide are attached to a small core structure. A central component defining the branched architecture is the core matrix which multimerizes dendrimic peptides to give them a cascade or pennant type of arrangement (Tam, J. Immunol. Method, 196: 17-32, 1996). The poly[Asp(NH—NH₂)]n or poly[Glu(NH—NH₂)]n peptides, or designed combination, can be transformed to poly[Asp(N₃)]n or poly[Glu(N₃)]n and then used as a core to which the multiple copies of peptide (antigen) are conjugated by fragment coupling via azide method. The core and peptides are synthesized and purified separately.

### 2. Convertion of the Hydrazide to Azide

Azides are obtained by reaction of suitably protected peptide hydrazides with nitrous acid. Nitrous acid may be generated in situ by reactions such as with alkyl nitrites, nitric salts, or tetrabutylammonium nitrite. The quantitative conversion of hydrazides into azides can be assessed by spraying spots with the hydrazide test solution.

Generally, the acylation step is carried out immediately after production of the azide without its isolation, at low temperature (0-5° C.) by maintaining the pH of the mixture between 7 and 8 with amines such as TEA and DIPEA.

For cyclization reactions using azides, due to the relatively low reactivity of the azides it is advisable to carry out the fragment couplings at the highest possible concentration, while cyclization reactions require the principle of high dilution to be applied. Isocyanate formation via Curtius rearrangement is one of the major side reactions which may occur in azide coupling steps. (Curtius, T. (1890) Ber. Dtsch. Chem. Ges. 23, 3023; Schnabel, E. (1962) Justus Liebigs Ann. Chem. 659, 168; Hofmann et al. (1960) J. Am. Chem. Soc. 82, 3715; Pattaroni et al. (1990) Int. J. Pept. Protein Res. 36, 401).

3. Cyclization by Amide Bond formation

While head to tail cyclization (coupling of the activated C-α-carboxylic group to N-α-amino group of the same peptide) of peptides can be achieved, the presence of reactive side chain carboxyl groups must be considered in the process.

According to the findings of the applicants and the teachings herein, a peptide may be prepared synthetically containing multiple carboxylic acid side chain resides (typically Asp or Glu) in which a single acid side chain is selectively converted to a hydrazide by hydrazinolysis, subsequently converted to the azide, and reacted with a free amine (the alpha amino group of the peptide). An example of the use of an internal hydrazide containing peptide of the invention in a cyclization reaction is shown in the following scheme:

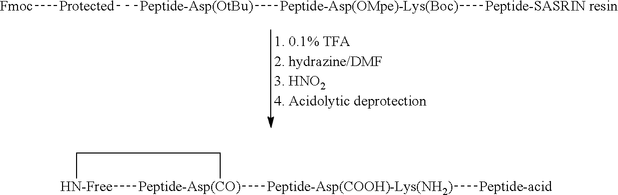

Using the teachings herein, a protected peptide containing an tert-butoxycarbonylated and a Me-pentylated aspartate ester reacted with hydrazine will selectively form a hydrazide at the Asp(OtBu)- position which will undergo reduction to the azide and cyclize with selectively with the alpha amine of the first residue.

Conversely, if a tail-to-head cyclization is desired, the synthesis of the protected peptide should include the use of OMpe esters at all side chain carboxylic acids to eliminate the formation of side chain hydrazides and direct the hydrazide and subsequent azide formation to the C-terminal carboxyl group. An example of such a process is shown in the below scheme.

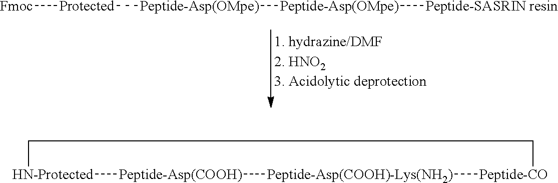

### 4. Convergent Synthesis

Lloyd-Williams at al. 1993 introduced the name “convergent” SPPS to describe fragment coupling using protected peptide segments via azide-amine acylation. This reaction can also be achieved with improved yield when the protected-peptide synthesis comprising carboxylated side chains is undertaken using OMpe esters according to the following scheme:

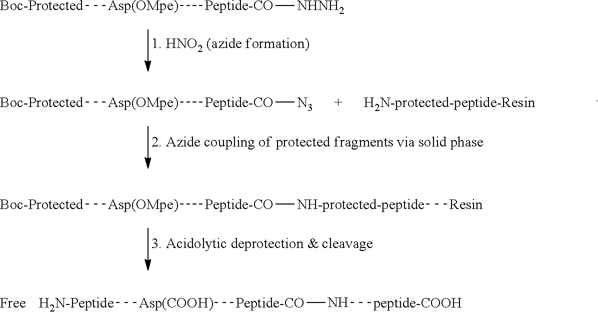

Alternatively, branched peptide synthesis can be achieved using a strategy that incorporates tert-butoxylated carboxylate esters were fragment joining is desired.

While having described the invention in general terms, the embodiments of the invention will be further disclosed in the following examples.

### EXAMPLE 1

### Attempt to Synthesize [GLP-1] [ [D-ALA², GLY³¹]-PEG₁₂]-GLY-N₂H₃ (II)

The human hormone, glucagon-like peptide-1 (GLP-1, SEQ ID NO: 1), is active in lowering blood glucose and therefore considerable effort has been focused on developing therapeutic agents which are GLP-1 analogs or mimic its biological activity and have suitable pharmacokinetic parameters such as longer serum half-life and reduced susceptibility to proteolytic degradation. As part of such research, it was desired to make a GLP-1 fragment suitable for linkage to other chemical moieties via hydrazone or azide coupling reactions. Boc-His(Boc)-D-Ala-Glu(OtBu)-Gly-Thr(tBu)-[Phe-Thr(Ψ^{Me-Me}pro)]-Ser(tBu)-Asp(OtBu)-Val-[Ser(tBu)-Ser(Ψ^{Me-Me}pro)]-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Gly-Arg(Pmc)-Gly-PEG₁₂-Gly-SASRIN™-resin (I), where PEG₁₂ is —NH—CH₂—CH₂—(O—CH₂—CH₂)₁₂—CO— and -AA-Thr(Ψ^{Me-Me}Pro) or -AA-Ser(Ψ^{Me-Me}Pro) are pseudoproline dipeptides (NovaBiochem), was synthesized by standard methods and subjected to hydrazinolysis.

Methods: The protected peptide-resin, (I), was prepared on an ABI 433A Peptide Synthesizer using SynthAssist 2.0 Version for Fmoc/HBTU chemistry by the Fastmoc 0.1 mM. Fmoc-Gly-SASRIN resin (139 mg, 1.10 mmol) (Bachem, substitution 0.79 mmol/g) was used in the synthesis. Boc-His(1-Boc)—OH (433.0 mg, 1 mM) (Bachem) (coupled as N-terminal amino acid derivative) was used in the first amino acid position in the sequence, starting numbering from the N-termini. Fmoc-D-Ala—OH (311.0 mg, 1 mM) (Bachem) was used for the second amino acid position in the sequence. Fmoc-Phe-Thr(Ψ^{Me-Me} pro)—OH (529.0 mg, 1 mM) (Novabiochem) was used for the sixth and seventh amino acid position in the sequence. Fmoc-Ser(But)-Ser(Ψ^{Me-Me} pro)—OH (511.0 mg, 1 mM) (Novabiochem) was used for the eleventh and twelfth amino acid position in the sequence. The O—(N-Fmoc-2-aminoethyl)-0′-(2-carboxyethyl)-undecaethyleneglycol (900 uL, 1 mM) (Novabiochem) was used in the thirty-second amino acid position in the sequence. The resin was washed with ethanol and dried overnight in vacuo. The final weight of the peptide-resin (I) was 0.48 g.

A previously described procedure (Mergler & Nyfeler, 1991) using either 10% hydrazine/DMF over 2 hours (A) or 20% hydrazine/DMF over 24 hours (B) was employed for the hydrazinolysis.

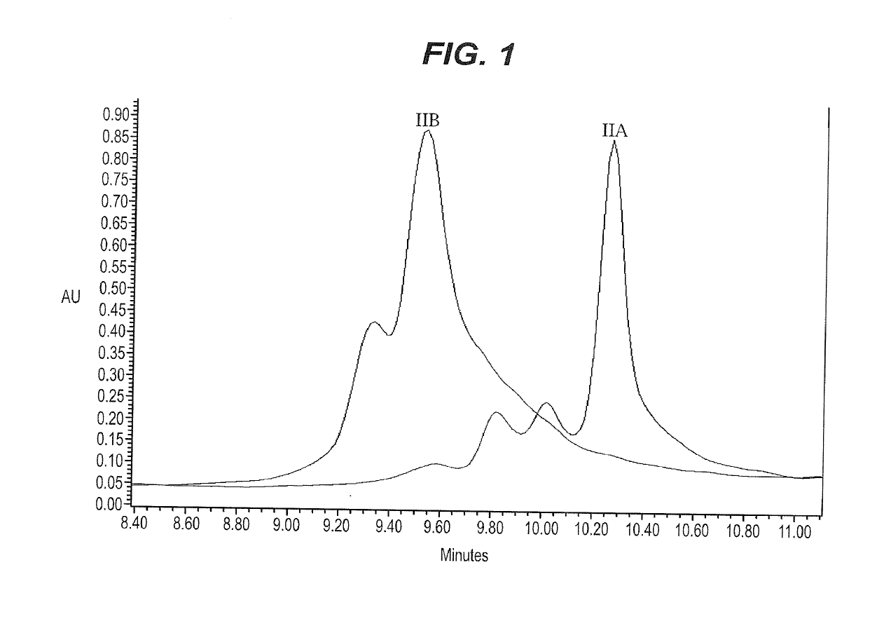

The protected peptide hydrazides were deblocked using an acidolytic cleavage mixture. An analytical RP HPLC and LC MS showed that two distinct products were obtained from the 24 hour reaction but not the 2 hour reaction (IIA and IIB, FIG. 1). LC-MS of the crude peptide mixture from the 2 H reaction gave a main peak with a mass of 4,027.1 Da, which corresponds to expected, C-terminal hydrazide (II) (calculated molecular weight: 4,026.5 Da):

HaEGTFTSDVSSYLEGQAAKEFIAWLVKGRG-PEG12-Gly-N₂H₃ (II)

### (where “a” =D-Ala)

However, the LC-MS of the crude peptide mixture from the 24 H reaction showed that it was a mixture of two products: (LC-MS: 4055.1) [M+28 Da] and (LC-MS: 4068.9) [M+42 Da].

The products from B were purified by preparative RP HPLC then analyzed by MS/MS and N-terminal sequencing. Samples were analyzed on the ABI 4700 Proteomics Analyzer. Product ion mass spectra were acquired in both metastable and collision-induced dissociation modes. The +28 Da and +42 Da modifications were determined to be the result of two or three +14 Da additions to acidic residues in the peptide, Glu¹⁵ and Asp⁹. A third site was deduced as Glu³, but compelling evidence was not obtained for this site. The reason Glu²¹ was not modified is not known (it is possible that was lost during HPLC purification). The automated Edman sequencing of each of the peptides fully corroborated the theoretical sequence. The discussed LC MS data suggested possible structure of the second peak in the 24 hour reaction to be:

H-a-E(+14 Da)-GTFTS-D(+14 Da)-VSSYL-E(+14 Da)-GQAAKEFIAWLVKGRG-PEG₁₂-Gly-N₂H₃ (IIB)

The -Glu(+14 Da)- or -Asp(+14 Da)- analyzed versus -Glu(CO—OH)- or -Asp(CO—OH)- also suggested that the —OH (17 Da) group in (IIB) was substituted by residue —X, with molecular weight of 31 Da: [31 Da] (—X)-17 Da] (—OH)]=[+14 Da].

Under the reaction conditions, X could be a methoxy group (—OCH₃, 31 Da) where the methyl group derives from the DMF, or a hydrazino- group (—NH—NH₂, 31 Da). The +14 Da modification of the Asp or Glu residues might be a result of methyl ester formation, -Asp(OCH₃)- [+14 Da] or -Glu(OCH₃)-(+14 Da), from the corresponding (tBu) esters. The presence of +14 Da side chain modified Glu and Asp residues may be explained as a result of Asp(NH—NH₂) (+14 Da) and Glu(NH—NH₂) (+14 Da) formation from the corresponding (tBu) esters.

To verify this hypothesis, the hydrazinolysis of protected peptide-SASRIN resin (I) was repeated under A and B conditions using DMF-d₇. If DMF was the methyl source in a trans-esterification reaction, then the labeled DMF would result in mass shift of +17 Da, versus +14 Da if the source was not the DMF or if a methyl group was not transferred.

Methods: The protected peptide-resin, Boc-His(Boc)-[D-Ala]-Glu(OtBu)-Gly-Thr(tBu)-[Phe-Thr(Ψ^{Me-Me}pro)]-Ser(tBu)-Asp(OtBu)-Val-[Ser(tBu)-Ser(Ψ^{Me-Me}pro]-Tyr(tBu)-Leu-Glu(OtBu)-Gly-Gln(Trt)-Ala-Ala-Lys(Boc)-Glu(OtBu)-Phe-Ile-Ala-Trp(Boc)-Leu-Val-Lys(Boc)-Gly-Arg(Pmc)-Gly-NH—CH₂—CH₂—(O—CH₂—CH₂)₁₂—CO-Gly-SASRIN Resin](I) was synthesized as described for (IIA) starting with Fmoc-Gly-SASRIN resin (131 mg, 1.03 mmol) (Bachem, Lot# 10001403, substitution 0.79 mmol/g). The final weight of the peptide-resin (I) was 0.550 g.

In order to evaluate whether a methyl ester was formed, hydrazinolysis was repeated using 20% hydrazine/DMF-d₇ over 2 hours or 24 hours. The experiment was repeated twice. The LC-MS analysis of the crude products are presented in Table 2. The LC-MS data found for crude products (protected peptides) isolated after the hydrazinolysis reactions are shown in Table 2.

TABLE 2

| | | M + | M + | M + | M + |
|---|---|---|---|---|---|
| | M Da | 14 Da | 28 Da | 42 Da | 56 Da |
| 2 hours | 4028.6 | 4042.4 | 4050.3 | 4071.9 | 4085.2 |
| 24 hours | — | 4040.2 | 4054.4 | 4068.3 | 4084.4 |
| 2 hours′ | — | 4042.3 | 4056.5 | 4070.6 | 4084.4 |
| 24 hours′ | — | 4042.0 | 4056.5 | 4070.6 | 4084.5 |

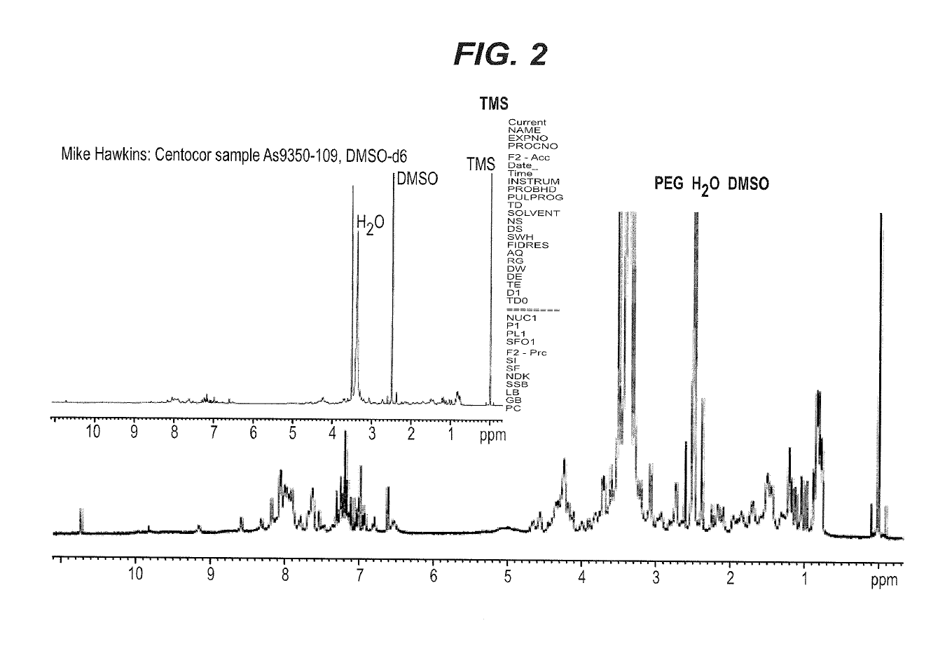

If DMF was the methyl source in a trans-esterification reaction, then the labeled DMF would result in mass shift of +17 Da, versus +14 Da if the source was not the DMF, for each methyl group transferred. MS data (intact molecular weight and sequence analysis) did not support the hypothesis of esterification with a methyl group from DMF. The -COOCD₃ has not been formed. The GLP-1[D-Ala², Gly³¹-PEG₁₂-Gly³²]—NH—NH₂ (IIB) was analyzed by ¹H-NMR spectroscopy for the presence of —OCH₃ signals; the spectra is shown in FIG. 2. No -OMe signal was evident at 3.3-3.5 ppm and did not support methyl esters formation at Asp or Glu residues.

### EXAMPLE 2

### Analysis of [GLP-1] [[D-ALA², GLY³¹]-PEG₁₂]-GLY-N₂H₃ [GLU(NH—NH₂)-3, ASP (NH—NH₂)-9, ASP(NH—NH₂)-15] (IIB)

The peptide-resin (I) (0.550 g) was swollen five minutes in DMF, filtered and transferred to a scintillation vial. The DMF swollen resin was mixed with 15 mL of 20% anhydrous hydrazine (Aldrich, Lot# 08513HD)/DMF and stirred over 24 h at ambient temperature. The resin was filtered off, washed with DMF (2×2 mL), then 700 mL of hot (about 70° C.) water was added to the filtrate and left to stand overnight. The white precipitate was filtered, washed with water (3×40 mL) and ethyl ether (3×40 mL) and then dried in vacuum to give 390 mg of white, crude, protected material. The protected product (385 mg) was deblocked using 20 mL of a cleavage mixture of TFA (20 mL), phenol (1.5 g), DTT (1.0 g), thioanisole (1.0 mL), TIS (1.0 mL), and water (1.0 mL) for two hours at ambient temperature. The resin was filtered off and the peptide was precipitated with precooled ethyl ether (400 mL), then filtered off and washed with ethyl ether. The crude peptide was dried in a vacuum to give 243 mg of white, crude, free product (LC-MS: 4,054.4 Da [M+28]+calculated mol. weight for IIA: 4,026.5). The crude peptide (51 mg, 63 mg, 72 mg and 49 mg) was purified in four injections by dissolving in 4.0 mL 6M Guanidine HCL and injecting in two Vydac C-18 columns (10 mm, 2.5×25 cm), using a gradient of 0-35% (80% acetonitrile/0.1% TFA in water) over 5 min and eluting on a gradient 35-60% (80% acetonitrile/0.1% TFA in water) over 60 min at a flow rate of 6 mL/min. Fractions were collected, analyzed by HPLC and the pure fractions pooled and lyophilized to give 47.3 mg (Fr. 1) of white product and two other side fractions (Fr. 1A: 24.7 mg; and Fr. 1B (14.2 mg).

In RP HPLC, Fr. 1, Fr. 1A and Fr. 1B, eluted as single, symmetrical peaks but capillary electrophoresis (CE) indicated the peaks area about 70%. The LC-MS analyses of the fractions are presented in Table 3. The LC-MS data found for fractions isolated after preparatory HPLC of the 24 H hydrazinolysis reaction gave the expected sequence and molecular weight was of IIA (4026.5 Da) but several derivatives in addition.

TABLE 3

| Fr./Batch (IIB) | M + 28 Da | M + 42 Da | M + 56 Da |
|---|---|---|---|
| Fr. 1A | 4055.0 | 4069.2 | — |
| Fr. 1 | 4055.1 | 4068.9 | 4084.5 |
| Fr. 1B | 4055.2 | 4069.3 | 4083.1 |

The differential HPLC retention of the 1A, 1 and 1B fractions, which are the same molecular mass could also be explained by the presence of diastereoisomers resulted from epimerization of Asp or Glu asymmetric carbons during transformation from tert-butyl esters to hydrazides.

For MS/MS, ion mass spectra (metastable dissociation), the samples were in 0.1% TFA at an estimated concentration of 2.0 mg/mL. The samples were diluted 1:10 in water, and then an additional 1:10 in CHCA matrix (10 mg/mL in 50:50:0.1 H2O/ACN/TFA). Samples were analyzed on the ABI 4700 Proteomics Analyzer in reflector mode from m/z 500-6000. Product ion mass spectra were acquired in both metastable and collision-induced dissociation modes.

N-terminal sequencing was performed as follows: aliquots of each of the samples (˜500 pmol) were blotted to PVDF membranes in ProSorb cartridges, and desalted with 500 μL of 0.1% TFA. Data was acquired for 30 cycles using standard cycles and conditions, and there was no addition of Biobrene for these analyses.

The results of MS/MS for all of the samples were characterized by a nearly complete set of yn ions (n=1-28), and except for slight differences in relative abundance, are virtually indistinguishable from each other. Ions characteristic of the C-terminal 16 residues are all found at the theoretical mass, indicating that the PEG-hydrazide modification was correctly synthesized. Ions for y17 through y22 are all shifted by +14 Da from predicted values, while ions for y23 through y28 are all shifted by +28 Da. These results indicate a +14 Da modification at Glu15 (yl7) and another +14 Da modification at Asp9 (y23). The third +14 Da modification site in the Fr. 1A sample must be located in 1-HAE-3. There were no ions observed which could define the location more specifically. Collision-induced dissociation MS/MS spectra (not shown) did not show any distinct differences in the low mass region of the spectra (immonium ions) that would imply modified His or Ala. It is reasonable to conclude, predicated on the +14 Da motifs already discerned, that the modification is on Glu³. This is partially corroborated by the reduced relative abundance of y28, as the cleavage that generates the y28 ion is no longer C-terminal adjacent to an acidic residue.

N-terminal sequencing was performed using an automated Edman sequencer for each of the peptides fully corroborated the theoretical sequence, except in cycle 15. During that cycle, no amino acid could be definitively called, and all subsequent cycles suffered from dramatically reduced signal intensity. This signal depression was also observed, albeit to a lesser extent, in cycle 8 for the modified aspartic acid. Cycles 9 through 14 rebounded somewhat in the integrated peak area of the principal amino acid detected

In summary, the +28 and +42 Da modifications to the anticipated final peptide-hydrazide (I) were determined to be the result of two or three +14 Da groups added to acidic residues in the peptide. The additions were determined to be principally at Glu¹⁵ and Asp⁹. The third site was deduced as Glu³, but compelling experimental evidence was not obtained for that site. No evidence was obtained for the differential retention of the 1 and 1B fractions, which are the same molecular mass. The C-terminal PEG-hydrazine (II) was found to be intact and no MS/MS based evidence could be obtained to elucidate any potential alterations to the C-terminus.

### EXAMPLE 3

### Preparation of [GLP-1] [[D-ALA², GLY³¹]-PEG₁₂]-GLY-N₂H₃ (II)

The peptide-resin (I) (0.189 g) was mixed with 5 mL of 10% hydrazine (anhydrous)/DMF and stirred over 2 hrs at ambient temperature. The resin was filtered off, washed with 1 mL of DMF, then 100 mL of hot (about 70° C.) water was added to the filtrate and left to stand overnight. The white precipitate was filtered, washed with water (3×20 mL) and ethyl ether (3×40 mL) and then dried in vacuum to give 126 mg of white material. The product (120 mg) was deprotected using a 10 mL of a cleavage mixture of TFA (20 mL), phenol (1.5 g), DTT (1.0 g), thioanisole (1.0 mL), TIS (1.0 mL), and water (1.0 mL) for two hours at ambient temperature. The resin was filtered off and the peptide was precipitated with precooled ethyl ether (400 mL), then filtered off and washed with ethyl ether. The crude peptide was dried in a vacuum to give 95 mg of white, crude product (IIA) (LC-MS: 4,028.0 Da, calculated mol. weight: 4,026.5). The crude peptide (IIA) (40 mg and 40 mg) was purified in two injections by dissolving in 3.0 mL of 25% acetic acid and injecting in two Vydac C-18 columns (10 mm, 2.5×25 cm), using a gradient of 0-30% (80% acetonitrile/0.1% TFA in water) over 5 min and eluting on a gradient 30-60% (80% acetonitrile/0.1% TFA in water) over 60 min at a flow rate of 6 mL/min. Fractions were collected, analyzed by HPLC and the pure fractions pooled and lyophilized to give 23 mg of white, pure product (II). LC MS: 4028.0 Da (calculated molecular weight: 4026.5 Da; CE gave a peak greater than 94%; HPLC showed a single peak which was [GLP-1] [[D-Ala², Gly³¹]-PEG₁₂]-Gly-N₂H₃ (II).

### EXAMPLE 4

### Synthesis of GLU(NH—NH₂)-ASP(NH—NH₂)-GLU(NH—NH₂)-ASP(NH—NH₂)-ALA-GLY-NH—NH₂ (IV)

A model hexapeptide containing two repeated Glu-Asp units was used to examine the relative stability and potential sequence dependence of the tert-butoxycarbonylated residues to hydrazinolysis. The protected, hexapeptide-SASRIN resin model with multiple glutamyl and aspartyl residues having the side-chains protected by tBu esters (III), was synthesized and reacted with 20% hydrazine/DMF mixture over 24 hours at ambient temperature, and after acidolytic removal of the Boc group analyzed by MS.

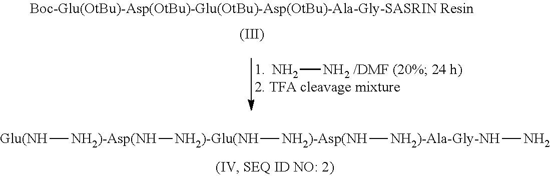

The LC MS analysis of the crude product gave a main peak with 705.3 Da mass, that was 56.7 Da (4×14 Da) higher, than calculated for Glu-Asp-Glu-Asp-Ala-Gly-NH—NH₂ (calculated molecular weight: 684.6 Da). Calculated molecular weight for IV is 704.6 Da and, thus, in good agreement with the identity of product IV. This result supports conclusion that the hydrazinolysis of tert-butyl esters within a protected peptide is not unique to the sequence of the GLP-1 analog (II, SEQ ID NO: 1).

### EXAMPLE 4A

### Hydrazinolysis Conditions

The hexapeptide (III) was reacted with 20% hydrazine or with 20% hydrazine hydrate/DMF over 24 hours at ambient temperature. The crude product was isolated and deblocked with TFA cleavage mixture using acidolytic cleavage mixture of 50% TFA/50% DCM/1% water for 1.5 hours at ambient temperature to give the crude peptide mixtures that were analyzed by LC-MS. The results are shown in Table 3. The calculated molecular weight for the final peptide Glu-Asp-Glu-Asp-Ala-Gly-NH—NH₂ is 648.6 Da. The LC-MS main peak corresponded to derivative (IV).

TABLE 3

| | M + 28 Da | M + 42 Da | M + 56 Da (100%) |
|---|---|---|---|
| Hydrazine | 677.5 | 691.5 | 705.5 |
| Hydrazine | 677.5 | 691.5 | 705.7 |
| monohydrate | | | |

Therefore, these results show that either condition anhydrous or hydrated hydrazinolysis of tert-butoxycarbonyl esters results in products having at least one additional derivatization. The mass units are consistant with the formation of 2, 3 or 4 additional 14 Da mass units which is consistent with the replacement of a carboxylic acid ester with a hydrazino group.

### EXAMPLE 5

### Synthesis of PHE-ASP(NH—NH₂)-LYS-ASP(COOH)-PHE-ALA-PHE-GLY-NH—NH₂ (VIIA)

To test the relative stability of protecting groups to hydrazinolysis, an octapeptide (SEQ ID NO: 3) having two non-adjacent aspartyl residues was designed and various combinations of protecting esters were used at the two positions to gauge relative stability to attack by hydrazine.

### OMpe and OtBu

Protected octapeptide-resin, Boc-Phe-Asp(OtBu)-Lys(Boc)-Asp(OMpe)-Phe-Ala-Phe-Gly-SASRIN-Resin (V), was prepared on an ABI 433 Peptide Synthesizer using SynthAssist 2.0 Version for Fmoc/HBTU/DIEA chemistry by the Fastmoc 0.25mM Monitoring Previous Peak software. Fmoc-Gly-SASRIN resin, (316 mg, 0.25 mmol) (Bachem D-1345, Lot#1001403, substitution 0.79 mmol/g) was used in the synthesis. Fmoc-Asp(OMpe (2-methyl-2—O-pentyl))—OH, (Novabiochem, 04-12-1259, Lot# A34320) was used in the fourth position. After synthesis the resin was washed with ethanol and vacuum dried overnight.

The peptide-resin was divided in half (˜230 mg) and each portion transferred to a small fitted reaction vessel and washed with DMF. Five mL of a 20% hydrazine (anhydrous), (Aldrich, 215155)/DMF was added to the reaction vessel and rotated for either 2 hrs or 24 hrs at ambient temperature. The supernatant was drained into a 250 Erlenmeyer flask containing 100 mL of 70° C. water while swirling and the reaction vessel was rinsed 2×0.5 mL DMF. The precipitated, protected peptide hydrazide mixtures from each reaction were allowed to cool to ambient temperature and was then placed overnight at 10° C. The peptide was filtered off, washed 3×20 mL water, 3×40 mL ethyl ether and vacuum dried in a desiccator. Analytical HPLC and LC/MS of the protected product mixtures were performed and are presented in the FIGS. 3 (2 H) and 4 (24 H) reactions, respectively. Of the four theoretically possible peptides (VIA-D), only three were identified (VIA, B, and D).

| | | MW (Da) |
|---|---|---|
| Species | Protected Peptide Structure | Protected |
| VIA | Boc-Phe-Asp(OtBu)-Lys(Boc)-Asp(OMpe)-Phe- | 1300.1 |
| | Ala-Phe-Gly-NH—NH₂ | |
| VIB | Boc-Phe-Asp(NH—NH₂)-Lys(Boc)-Asp(OMpe)- | 1259.49 |
| | Phe-Ala-Phe-Gly-NH—NH₂ | |
| VIC | Boc-Phe-Asp(OtBu)-Lys(Boc)-Asp(NH—NH₂)- | 1230.4 Da |
| | Phe-Ala-Phe-Gly-NH—NH₂ | |
| VID | Boc-Phe-Asp(NH—NH₂)-Lys(Boc)- | 1188.33 |
| | Asp(NH—NH₂)-Phe-Ala-Phe-Gly-NH—NH₂ | |

The crude peptide from the 2 hours hydrazinolysis and 24 hours hydrazinolysis were analyzed by LC-MS (FIG. 3). By HPLC, the main product after 2 hours of reaction) was VIB (1,259 Da, 56%) with VIA (1300 Da, 26%) and VID (1188 Da, 18%). After 24 hours hydrazinolysis of (V), the main product was VID (1188 Da, 84%) with VIB (1259 Da, 16%) as the secondary product ((FIG. 4). Thus, these data demonstrate the higher stability of OMpe ester over OtBu ester protecting group against reaction with hydrazine

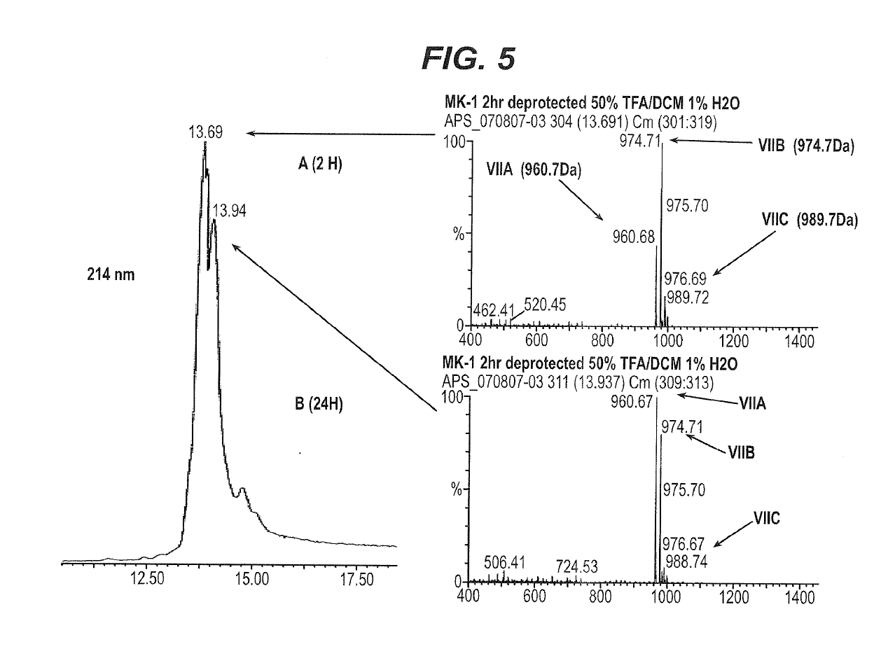

The two batches of protected peptide hydrazides were deprotected using acidolytic cleavage mixture of 50% TFA/50% DCM/1% water for 1.5 hours at ambient temperature. The peptides were precipitated with methyl t-butyl-ether, placed on ice for 1 hr, filtered and dried under vacuum overnight. Yield of white solids: 87 mg for the 2 H reaction and 86 mg from the 24 H hydrazinolysis. The HPLC and LC-MS analysis showed that each of crude product mixtures contained the all three of the corresponding possible free peptides (VIIA, B, and D). FIG. 5 shows the LC-MS of the crude the deblocked reaction mixture from the 2 H hydrazinolysis reaction.

| | | MW (Da) |
|---|---|---|
| Species | Deprotected Peptide Structure | Deblocked |
| VIIA | Phe-Asp(COOH)-Lys-Asp(COOH)-Phe-Ala-Phe- | 960 |
| | Gly-NH—NH₂ | |
| VIIB | Phe-Asp(NH—NH₂)-Lys-Asp(COOH)-Phe- | 974 |
| | Ala-Phe-Gly-NH—NH₂ | |
| VIID | Phe-Asp(NH—NH₂)-Lys-Asp(NH—NH₂)- | 988 |
| | Phe-Ala-Phe-Gly-NH—NH₂ | |

Portions of the remaining, crude mixture of deprotected peptide from the 2 H hydrazinolysis reaction (38 mg and 39 mg) was purified in two injections by dissolving in 4.0 mL 25% acetic acid and injecting in two Vydac C-18 columns (10 mm, 2.5×25 cm), using a gradient of 0-20% (80% acetonitrile/0.1% TFA in water) over 5 min and eluting on a gradient of 20-45% (80% acetonitrile/0.1% TFA in water), over 60 min at a flow rate of 6 ml/min. Fractions were collected, analyzed by analytical HPLC and LC/MS and the pure fractions pooled and lyophilized to give 18 mg of white product (LC/MS: 974.6 Da, calculated mol. weight: 974.0 Da)

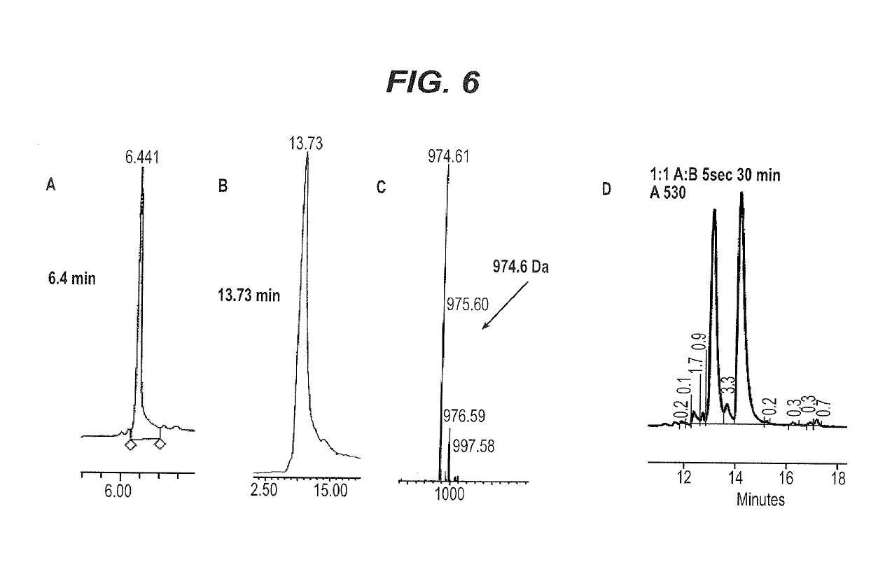

Capillary electrophoresis indicated peak areas of 40% and 52% respectively at pH 2.5. The analytical data for the Phe-Asp(NH—NH₂)-Lys-Asp(COOH)-Phe-Ala-Phe-Gly-NH—N2 (VIIB) are shown in FIG. 6.

The remaining crude peptide hydrazide from the 24 H hydrazinolysis reaction (42 mg and 33 mg) was purified in two injections by dissolving in 4.0 mL 25% acetic acid and injecting in two Vydac C-18 columns (10 mm, 2.5×25 cm), using a gradient of 0-20% (80% acetonitrile/0.1% TFA in water) over 5 min and eluting on a gradient of 20-45% (80% acetonitrile/0.1% TFA in water), over 60 min at a flow rate of 6 ml/min. Fractions were collected, analyzed by HPLC and LC/MS and the pure fractions pooled and lyophilized to give 26 mg of white product (LC/MS: 988.7 Da, calculated mol. weight: 988.0 Da)

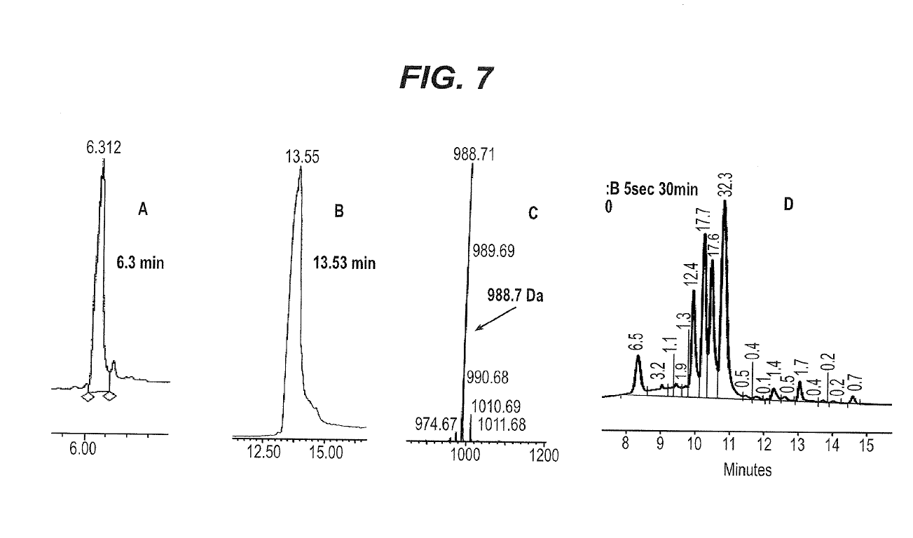

The analytical data for the of Phe-Asp(NH—NH₂)-Lys-Asp(NH—NH₂)-Phe-Ala-Phe-Gly-NH—N₂ (VIID) are shown in FIG. 7. This product shows expected MS data, single peak by HPLC, however the presence of four peaks in CE indicates the presence of four diasteroisomers.

### OPhiPr and OtBu

The same sequence was used to synthesis protected-peptide Boc-Phe-Asp(OtBu)-Lys(Boc)-Asp(O—2-PhiPr)-Phe-Ala-Phe-Gly-SASRIN-Resin (VI) which was similarly subjected to NH₂NH_{2/}DMF (20%) for either 2 h (A) or 24 h (B).

After 2 hours hydrazinolysis two products were found, 78% was the 1188.33 Da species (VID) and 22% was Boc-Phe-Asp(OtBu)-Lys(Boc)-Asp(NH—NH₂)-Phe-Ala-Phe-Gly-NH—NH₂ (VIC).

After 24 hours of hydrazinolysis, as with the other pair of protecting groups, a single product (VID) (LC MS: 1188.3 Da) was found.

Thus, these data indicate that the -Asp(OMpe)- residue is more stable than corresponding -Asp(OtBu)- ester in reaction with 20% hydrazine/DMF and the -Asp(OtBu)-ester was more stable in reaction with hydrazine than -Asp(O-PhiPr)

What is to be claimed:

1. A method of preparing a protected peptide hydrazide, wherein the hydrazide is not at the N- or C-terminus of the peptide and wherein the hydrazide is a beta- or gamma hydrazide of an Asp or Glu residue, comprising
a) preparing a peptide by chemical synthesis using an ester of Asp or Glu selected from the group consisting of OMpe, OtBu, and OPhiPr at the position in the peptide of the intended beta- or gamma hydrazide,
b) contacting the protected peptide with hydrazine under conditions where the ester is subjected to nucelophilic attack by hydrazine, and
c) purifying the peptide.

2. The method as in claim 1, of preparing a protected peptide hydrazide, additionally comprising the step of selecting the position of the hydrazide from among the residues comprising carboxy side chains.

3. The method as in claim 2, of preparing a protected peptide hydrazide, additionally comprising the step of choosing the ester at the selected residue to be more labile to hydrazine than the esters of other carboxy side chain residues.

4. The method as in claim 3, of preparing a protected peptide hydrazide, where the more labile ester comprises OtBu and the esters of the other carboxy side chain residues comprise OMpe.

5. The method as in claim 3, of preparing a protected peptide hydrazide, where the more labile ester comprises OPhiPr and the esters of the other carboxy side chain residues comprise OMpe.

6. The method as in claim 3, of preparing a protected peptide hydrazide, where the more labile ester comprises OPhiPr and the esters of the other carboxy side chain residues comprise OtBu.

7. The method as in claim 1, of preparing a protected peptide hydrazide, additionally comprising
d) converting the side chain hydrazide group to azide.

8. A method of making a derivatized peptide wherein the peptide made by the method of claim 1 is subjected to the additional steps of:
d) reacting the peptide with a derivatizing moiety comprising a reactive carbonyl group.

9. A method of making a derivatized peptide wherein the peptide made by the method of claim 1 is subjected to the additional steps of:
d) converting the hydrazide to an azide, and
e) reacting the peptide with a derivatizing moiety comprising an carboxyl functional group.

10. The method of making a derivatized peptide of claim 9, where the peptide is derivatized with a derivative selected from the group consisting of a peptide, a chelating group, a chromophore, a fluorophore, and a bioactive agent.

11. The method of preparing a protected peptide hydrazide of any of claims 1-10, wherein the hydrazinolysis is performed using 20% hydrazine.

12. A method of making a cyclized peptide wherein the peptide made by the method of claim 1 is subjected to the additional steps of
d) treatment with nitrite selected from the group consisting of alkyl nitrite, nitrite salts, and tetrabutylammonium nitrite.

13. A peptide prepared by the method of claim 1 having an internal beta- or gamma-hydrazide.

14. The use of a peptide of claim 1 as an immunogen.


SEQUENCE LISTING (3 sequences)

SEQ ID NO: 1 - protein, 31 aa, Homo sapiens
  VARIANT 2, 31: Xaa = Any Amino Acid
HXEGTFTSDVSSYLEGQAAKEFIAWLVKGRX

SEQ ID NO: 2 - protein, 6 aa, Artificial Sequence
  Repeated Glu-Asp units
EDEDAG

SEQ ID NO: 3 - protein, 8 aa, Artificial Sequence
  Synthetic peptide of no know function
FDKDFAFG